(19) United States
(12) Patent Application Publication
(10) Pub. No.: US 2016/0108391 A1
(43) Pub. Date: Apr. 21, 2016

(54) REAGENT COMPOSITIONS HAVING PYRIDINE-CARBOXYLIC ACID-STABILIZED ENZYMES, AS WELL AS METHODS OF MAKING AND USING THE SAME

(71) Applicant: Roche Diabetes Care, Inc., Indianapolis, IN (US)

(72) Inventors: Gabriele Chemnitius, Mannheim (DE); Thomas Nagel, Mannheim (DE); Otto Gaa, Worms (DE); Karl Recht, Buerstadt (DE)

(73) Assignee: ROCHE DIABETES CARE, INC., Indianapolis, IN (US)

(21) Appl. No.: 14/936,389

(22) Filed: Nov. 9, 2015

Related U.S. Application Data
(63) Continuation of application No. PCT/EP2014/059418, filed on May 8, 2014.

(30) Foreign Application Priority Data
May 8, 2013 (EP) 13167046.5

(51) Int. Cl. C12N 9/96; C12N 9/04; C12Q 1/32

(52) U.S. Cl. CPC C12N 9/96; C12Q 1/32; C12N 9/0006; C12Y 101/9901

(57) ABSTRACT

Dry reagent compositions are disclosed that include one or more of an enzyme such as a dehydrogenase, a redox cofactor, an agent capable of eliciting at least one measurable change in a property of an indicator reagent in the presence of redox equivalents, an indicator reagent, and a pyridine-carboxylic acid, derivative or salt thereof. Also provided are diagnostic test elements including the reagent compositions for use in body fluid analysis. Further provided are methods of making test elements, as well as methods of body fluid analysis with such test elements. In the compositions, the pyridine-carboxylic acid, derivative or salt thereof attenuates, reduces or prevents a decrease of enzymatic activity of the at least one dehydrogenase under dry and/or humid conditions.

### CROSS-REFERENCE TO RELATED APPLICATIONS

This patent application is a continuation of Int'l Patent Application No. PCT/EP2014/059418 (filed 8 May 2014), which claims priority to and the benefit of EP Patent Application No. 13167046.5 (filed 8 May 2013). Each patent application is incorporated herein by reference as if set forth in its entirety.

### TECHNICAL FIELD

This disclosure relates generally to chemistry and medical diagnostics, and more particularly, it relates to reagent compositions for use in diagnostic test elements, as well as methods of making and using the same, where the enzymes in the reagent compositions, especially dry reagent compositions, are stabilized by a pyridine-carboxylic acid, derivative or salt thereof.

### BACKGROUND

Diagnostic test elements frequently are used in near-patient applications. Therefore, the test elements must be robust with respect to handling and storage. This applies, in particular, to the test chemistry of the test elements. See, Hönes et al. (2008) Diabetes Technol. Therap. 10:S10-S26.

Many diagnostic test elements, however, are based on a rather complex enzyme test chemistry present on the test element. For example, such test elements include a carrier and a detection layer, where the detection layer usually contains enzymes. It is decisive for the proper function of the test elements that these enzymes remain biologically active during storage and upon treatment. Since calibration for an individual measurement is usually not possible, the test elements are normally calibrated batch-wise. The calibration information for a batch of test elements is stored and used for each test element of the batch regardless of individual differences in treatment and storage.

Unfortunately, pretreatments of the test elements and storage conditions can severely affect enzyme activity. For example, heat treatment, during either the manufacture or the storage of the test elements, can denature the enzymes such that the overall enzymatic activity present on the test elements is significantly reduced which, in turn, will result in incorrect test results when such test elements are used. Similarly, many enzymes are sensitive with respect to oxidation processes, which also result in denaturation and irreversible enzyme inactivation. Moreover, many enzymes on test elements are present, at least during the time of storage, in a solvent-free environment that may further promote oxidation processes. Furthermore, the detection layer may include additional components that facilitate oxidation processes, such as redox cofactors and other redox-relevant components and the like.

The problem of preserving enzymatic activities under such unfavorable conditions, however, applies not only to test elements but also to many enzyme preparations that are provided and stored in essentially solvent-free form, such as freeze-dried preparations.

Various so-called compatible solutes (i.e., low molecular weight compounds of different chemical classes such as sugars, polyols, free amino acids, amino acid derivatives, amines and sulphur analogs, sulfate esters, short peptides, and cyclic 2,3-diphosphoglycerate) have been investigated for their preservative properties for proteins in solution and under dry conditions. See, Arakawa & Timasheff (1985) Biophys. J. 47:411-414; Lippert & Galinski (1992) Appl. Microbiol. Biotechnol. 37:61-65; Göller & Galinski (1999) J. Mol. Catal. B Enzymatic 7:37-45; Lentzen & Schwarz (2006) Appl. Microbiol. Biotechnol. 72:623-634; Int'l Patent Application Publication No. WO 2007/002657; and US Patent Application Publication No. 2010/0255120.

Stabilization of glucose oxidase by compatible solutes like ectoine and hydroxyectoine has been previously reported in electrochemical test elements for detecting glucose in solution. See, Int'l Patent Application Publication No. WO 2007/097653; and Loose (2006) Proceedings of the 24th IASTED Int'l Multi-Conference Biomedical Engineering, 167-173 (Innsbruck, AT). However, glucose oxidase is known to be a rather stable enzyme with respect to oxidative stress and heat. On the other hand, pyridine-carboxylic acids and derivatives thereof have been reported to be insufficient to effectively prevent inactivation of glucose dehydrogenase in solution. See, Hachisuka et al. (1967) J. Biochem. 61:659-661. Dehydrogenases in general and, in particular, glucose dehydrogenases are rather sensitive enzymes for oxidative stress and heat treatment. Despite this limitation, they are important diagnostic tools.

For the foregoing reasons, there is a need for preserving enzymatic activity of storage- and temperature-sensitive enzymes used for diagnostic applications and, in particular, for the class of dehydrogenases such as glucose dehydrogenase.

### BRIEF SUMMARY

An inventive concept described herein includes attenuating, reducing or preventing decreases in enzymatic activity of at least one enzyme in reagent compositions under dry and/or humid conditions. This inventive concept is achieved by adding a pyridine-carboxylic acid, derivative or salt thereof to reagent compositions during manufacture and/or storage. This inventive concept can be incorporated into exemplary compositions, test elements and methods as described herein and in more detail below.

For example, reagent compositions are provided that include one or more of the following: at least one dehydrogenase, at least one redox cofactor, at least one agent capable of eliciting a change in at least one measurable property of an indicator reagent in the presence of redox equivalents, at least one indicator reagent; and at least one pyridine-carboxylic acid, derivative or salt thereof.

In some instances, the at least one dehydrogenase can be a lactate dehydrogenase, a glucose dehydrogenases, an alcohol dehydrogenase, a L-amino acid dehydrogenase, a glycerin dehydrogenase, a malate dehydrogenase, a 3-hydroxybutyrate dehydrogenase, or a sorbitol dehydrogenase.

In some instances, the at least one redox cofactor can be a pyrrolo quinoline quinone (PQQ) cofactor, a nicotinamide-adenine-dinucleotide (NAD) cofactor or a derivative thereof, or a flavine cofactor such as flavin-adenine-dinucleotide (FAD).

In some instances, the at least one agent capable of eliciting a change in at least one measureable property of an indicator reagent in the presence of redox equivalents can be a diaphorase such as a lipoamide dehydrogenase or a NADH dehydrogenase; a phenazine such as phenazinethosulfate, phenazinmethosulfate, 1-(3-carboxypropoxy)-5-ethylphenaziniumtrifluoromethansulfonate or 1-methoxyphenazinmethosulfate; a nitrosoaniline such as [(4-nirosophenyl)imino]dimethanol-hydrochloride; and a chinone such as phenanthrenechinone, phenanthrolinchinone or benzo[h]-chinolinchinone.

In some instances, the at least one indicator reagent can be a heteropoly acid such as 2,18-phosphoromolybdenic acid; or chinones such as resazurine, dichlorophenolindophenole and/or tetrazolinum salts. In other instances, the at least one indicator reagent can be a fluorophore such as flavin nucleotides or nicotinamide-adenine dinucleotides. In particular instances, the agent and indicator reagent may be represented in the reagent compositions by the same chemical entity.

In some instances, the at least one pyridine-carboxylic acid, derivative or salt thereof can be pyridine-3-carboxylic acid, pyridine-4-carboxylic acid, pyridine-2-carboxylic acid, or a sodium salt or a potassium salt thereof.

Diagnostic test elements also are provided, where such test elements include a reagent composition as described herein, and where the reagent composition is a dry reagent composition applied to or incorporated into a carrier of the diagnostic test elements.

In view of the foregoing, methods also are provided for making test elements as described herein. In some instances, the methods can include the steps of applying a reagent composition as described above in a solvent to a test field on a carrier; and removing the solvent from the reagent composition.

In other instances, the methods can include the steps of applying a first composition including at least one dehydrogenase, at least one redox cofactor, at least one indicator reagent, and at least one pyridine-carboxylic acid, derivative or salt thereof in a solvent to a test field on the carrier; removing the solvent from the composition to form a first layer; applying a second composition including at least one agent capable of eliciting a change in at least one measurable property of an indicator reagent in the presence of redox equivalents and at least one pyridine-carboxylic acid, derivative or salt thereof in a solvent on the first layer; and removing the solvent from the second composition to form a second layer.

In other instances, the methods can include the steps of applying a first composition including at least one dehydrogenase, at least one redox cofactor, at least one indicator reagent and at least one pyridine-carboxylic acid, derivative or salt thereof in a solvent to a test field on a carrier; removing the solvent from the first composition to form a first layer; applying a second composition including at least one redox cofactor, at least one agent capable of eliciting a change in at least one measurable property of an indicator reagent in the presence of redox equivalents and at least one indicator reagent in a solvent on the first layer; and removing the solvent from the second composition to form a second layer.

In other instances, the methods can include the steps of applying a first composition including at least one dehydrogenase, at least one indicator reagent and at least one pyridine-carboxylic acid, derivative or salt thereof in a solvent to a test field on a carrier; removing the solvent from the first composition to form a first layer; applying a second composition including at least one redox cofactor at least one agent capable of eliciting a change in at least one measurable property of an indicator reagent in the presence of redox equivalents at least one indicator reagent and at least one pyridine-carboxylic acid, derivative or salt thereof in a solvent on the first layer; and removing the solvent from the second composition to form a second layer.

In other instances, the methods can include the steps of applying a first composition including at least one agent capable of eliciting a change in at least one measurable property of an indicator reagent in the presence of redox equivalents, at least one indicator reagent and at least one pyridine-carboxylic acid, derivative or salt thereof in a solvent to a test field on a carrier; removing the solvent from the first composition to form a first layer; applying a second composition including at least one dehydrogenase, at least one redox cofactor, at least one indicator reagent and at least one pyridine-carboxylic acid, derivative or salt thereof in a solvent on the first layer; and removing the solvent from the second composition to form a second layer.

In other instances, the methods can include the steps of applying a first composition including at least one redox cofactor, at least one agent capable of eliciting a change in at least one measurable property of an indicator reagent in the presence of redox equivalents, at least one indicator reagent and at least one pyridine-carboxylic, derivative or salt thereof in a solvent to a test field on a carrier; removing the solvent from the first composition to form a first layer; applying a second composition including at least one dehydrogenase, at least one redox cofactor, at least one indicator reagent and at least one pyridine-carboxylic acid, derivative or salt thereof in a solvent the first layer; and removing the solvent from the second composition to form a second layer.

In other instances, the methods include the steps of applying a first composition including at least one redox cofactor, at least one agent capable of eliciting a change in at least one measurable property of an indicator reagent in the presence of redox equivalents, at least one indicator reagent and at least one pyridine-carboxylic acid, derivative or salt thereof in a solvent to a test field on a carrier; removing the solvent from the first composition to form a first layer; applying a second composition including at least one dehydrogenase, at least one indicator reagent and at least one pyridine-carboxylic acid, derivative or salt thereof in a solvent on the first layer; and removing the solvent from the second composition to form a second layer.

Also provided are methods of determining an analyte concentration or presence that use the test elements described herein. The methods can include a step of contacting a test element as described herein with a body fluid sample having or suspected of having the analyte of interest under conditions suitable for transforming the at least one enzyme of the reagent composition to a reconstituted state.

The methods also can include a step of measuring a change in at least one measurable property of an indicator reagent in the wetted reagent composition, whereby a presence or an amount of the analyte in the body fluid sample is determined.

Also provided are methods of maintaining enzyme activity of a reagent composition under dry or humid conditions. The methods can include a step of contacting at least one pyridine-carboxylic acid, derivative or salt thereof with a dehydrogenase under dry or humid conditions. In some instances, the contact can be during manufacture of test elements when a solvent is being removed from a reagent composition or when maintaining the test elements in a dry condition.

These and other advantages, effects, features and objects of the inventive concept will become better understood from the description that follows. In the description, reference is made to the accompanying drawings, which form a part hereof and in which there is shown by way of illustration, not limitation, embodiments of the inventive concept.

These and other advantages, effects, features and objects of the inventive concept will become better understood from the description that follows. The description of exemplary embodiments is not intended to limit the inventive concept to the particular forms disclosed, but on the contrary, the intention is to cover all modifications, equivalents and alternatives falling within the spirit and scope of the inventive concept as defined by the embodiments above and the claims below. Reference should therefore be made to the embodiments above and claims below for interpreting the scope of the inventive concept.

### BRIEF DESCRIPTION OF THE DRAWINGS

The advantages, effects, features and objects other than those set forth above will become more readily apparent when consideration is given to the detailed description below. Such detailed description makes reference to the following drawings, wherein:

FIG. 1 shows a stabilizing effect of nicotinic acid derivatives on glucose dehydrogenase, especially the remaining activity of glucose dehydrogenase mutant 2 (ordinate) after storage for 3 and 9 weeks at 45° C. (abscissa) in an exemplary reagent composition as described herein (coating on test element) when compared to the remaining activity after storage at 4° C. (=100%).

FIGS. 2a-b show glucose concentration vs. readings from test elements stored at various temperatures normalized against readings from test elements stored at 4° C. FIG. 2a shows test elements produced with 1 g nicotinic acid per 100 g first layer coating dispersion; whereas FIG. 2b shows test elements produced without stabilizer added to the first layer coating dispersion.

FIGS. 3a-b show a stabilizing effect of nicotinic acid at 35° C. under dry and under humid storage conditions, especially a time course of remaining activity stored in a composition as described herein (coating on test strips) at 35° C. under humid (FIG. 3a) or under dry (FIG. 3b) conditions when compared to the residual activity after storage at 4° C. and dry conditions (=100%).

Corresponding reference characters indicate corresponding parts throughout the several views of the drawings.

While the inventive concept is susceptible to various modifications and alternative forms, exemplary embodiments thereof are shown by way of example in the drawings and are herein described in detail. It should be understood, however, that the description of exemplary embodiments that follows is not intended to limit the inventive concept to the particular forms disclosed, but on the contrary, the intention is to cover all advantages, effects, features and objects falling within the spirit and scope thereof as defined by the embodiments described herein and the claims below. Reference should therefore be made to the embodiments described herein and claims below for interpreting the scope of the inventive concept. As such, it should be noted that the embodiments described herein may have advantages, effects, features and objects useful in solving other problems.

### DESCRIPTION OF EXEMPLARY EMBODIMENTS

The reagent compositions, test elements and methods now will be described more fully hereinafter with reference to the accompanying drawings, in which some, but not all embodiments of the inventive concept are shown. Indeed, the reagent compositions, test elements and methods may be embodied in many different forms and should not be construed as limited to the embodiments set forth herein; rather, these embodiments are provided so that this disclosure will satisfy applicable legal requirements as illustrated herein being contemplated as would normally occur to one of skill in the art.

Likewise, many modifications and other embodiments of the reagent compositions, test elements and methods described herein will come to mind to one of skill in the art to which the disclosure pertains having the benefit of the teachings presented in the foregoing descriptions and the associated drawings. Therefore, it is to be understood that the reagent compositions, test elements and methods are not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the disclosure pertains. Although any methods and materials similar to or equivalent to those described herein can be used in the practice or testing of the reagent compositions, test elements and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.” Likewise, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. For example, the expressions “A has B,” “A comprises B” and “A includes B” may refer both to a situation in which, besides B, no other element is present in A (i.e., a situation in which A solely and exclusively consists of B) or to a situation in which, besides B, one or more further elements are present in A, such as element C, elements C and D, or even further elements.

Overview

Reagent compositions, especially dry reagent compositions, are provided that improve/increase long-term stability of enzyme activity even under humid conditions. Advantageously, the work described herein shows that a reduction of enzymatic activity that can occur during manufacture and/or storage of complex dry reagent compositions can be significantly attenuated, reduced or prevented by adding to the reagent composition a pyridine-carboxylic acid, derivative or salt thereof as specified herein. This finding is surprising since pyridine-carboxylic acids and derivatives thereof have been reported to be insufficient to effectively prevent inactivating glucose dehydrogenase in solution. See, e.g., Hachisuka et al. (1967), supra. Moreover, it is also surprising that the preservative effect occurs even under the redox-sensitive conditions present in the rather complex, solvent-free reagent compositions described herein. In particular, the enzymatic activity present in a dry reagent composition as described herein except for the pyridine-carboxylic acid, derivative or salt thereof was found to decrease during storage under dry or humid conditions and at temperatures above 4° C. However, significantly higher enzymatic activities could be maintained when a pyridine-carboxylic acid, derivative or salt thereof was present in the reagent composition. A further advantage of the reagent compositions described herein is that the pyridine-carboxylic acid, derivative or salt thereof does not interfere with a detection system for a measurable property. In particular, it does not interfere with the measurable signals generated and does not impair the stability or function of the indicator reagent, the redox cofactor, or the agent capable of eliciting the at least one measurable change. Moreover, the enzymatic conversion and conversion rates are not impaired by the pyridine-carboxylic acid, derivative or salt thereof.

As used herein, “composition” or “reagent composition” means a mixture including compounds as specified herein. One of skill in the art understands that the composition may include additional components such as, for example, buffer components (e.g., phosphate buffered saline, Tris buffer, citrate buffer, glycerine phosphate buffer or Good's buffer) or other salts, detergents, or the like, including the components as specified hereinbelow. Likewise, and as used herein, “dry composition” or “dry reagent composition” means that the composition is essentially free of a solvent or a mixture of solvents.

As used herein, “essentially free” means that at least about 85%, at least about 90%, at least about 91%, at least about 92%, at least about 93%, at least about 94%, at least about 95%, at least about 96%, at least about 97%, or at least about 98% of a solvent or solvent mixture that was originally present in a solution of the composition has been removed from the composition. Accordingly, it is envisaged that the solvent or solvent mixture is present in the dry reagent compositions as described herein in an amount of up to about 15%, up to about 10%, up to about 9%, up to 8%, up to about 7%, up to about 6%, up to about 5%, up to about 4%, up to about 3%, or up to 2%. Alternatively stated, a dry reagent composition can include water in an amount of up to about 15%, up to about 10%, up to about 9%, up to 8%, up to about 7%, up to about 6%, up to about 5%, up to about 4%, up to about 3%, or up to about 2%. Methods of determining residual water are known in the art. Here, for example, residual water in a dry reagent composition as described herein can be determined using a phosphorus pentoxid sensor and a method as described in Int'l Patent Application Publication No. WO 1993/012418. The aforementioned percentage values and the other percentage values referred to herein that are used to define amounts refer to percent by weight (w/w). As such, the dry reagent compositions as described herein generally are a solid composition under normal conditions (i.e., under room temperature and normal pressure).

Reagent Compositions

Reagent compositions, such as dry reagent compositions, incorporating the inventive concept can include one or more of the following: (1) at least one dehydrogenase, (2) at least one redox cofactor, (3) at least one agent capable of eliciting a change in at least one measurable property of an indicator reagent in the presence of redox equivalents, (4) at least one indicator reagent; and (5) at least one pyridine-carboxylic acid, derivative or salt thereof.

One component of the reagent compositions therefore is at least one dehydrogenase. As used herein, “dehydrogenase” means a polypeptide that is capable of catalyzing an oxidation or a reduction of a substrate by transferring hydrides (H⁻) as redox equivalents to or from an acceptor molecule such as a redox cofactor as referred to herein elsewhere. Dehydrogenases can depend on a redox cofactor (also called a co-enzyme), such as pyrrolo quinoline quinone (PQQ), nicotinamide-adenine-dinucleotide (NAD) or a derivative thereof, or a flavine cofactor such as flavin-adenine-dinucleotide (FAD) or flavine mononucleotide (FMN).

Examples of dehydrogenases include, but are not limited to, lactate dehydrogenase (EC number 1.1.1.27 or 1.1.1.28), glucose dehydrogenases, alcohol dehydrogenase (EC number EC number 1.1.1.1 or 1.1.1.2), L-amino acid dehydrogenase (EC number 1.4.1.5), glycerin dehydrogenase (EC number 1.1.1.6), malate dehydrogenase (EC number 1.1.1.37), 3-hydroxybutyrate dehydrogenase (EC number 1.1.1.30), or sorbitol dehydrogenase (EC number 1.1.1.14).

In some instances, the dehydrogenase is a glucose dehydrogenase. Examples of glucose dehydrogenases include, but are not limited to, glucose dehydrogenase (EC number 1.1.1.47), quinoprotein glucose dehydrogenase (EC number 1.1.5.2) such as pyrrolo quinoline quinone (PQQ)-dependent glucose dehydrogenase (EC number 1.1.5.2), glucose-6-phospate dehydrogenase (EC number 1.1.1.49), nicotinamide adenine dinucleotide (NAD)-dependent glucose dehydrogenase (EC number 1.1.1.119) and flavin adenine dinucleotide (FAD)-dependent glucose dehydrogenase (EC number 1.1.99.10) or enzymatically active mutants thereof.

Enzymatically active mutants of dehydrogenases can be obtained by substituting, adding or deleting one or more amino acids from a known amino acid sequence for the aforementioned wild-type dehydrogenases. One such mutant is a PQQ-dependent glucose dehydrogenase having improved substrate specificity when compared to its wild-type counterpart. See, e.g., U.S. Pat. Nos. 7,132,270 and 7,547,535. Other mutants are known and described in Baik et al. (2005) Appl. Environ. Microbiol. 71:3285-3293; Vasquez-Figuera et al. (2007) ChemBioChem. 8:2295-2301; and Int'l Patent Application Publication No. WO 2005/045016.

In certain instances, the glucose dehydrogenase mutant has a mutation at least at amino acid positions 96, 170 and/or 252. See, e.g., Int'l Patent Application Publication No. WO 2009/103540. Particular amino acid substitutions are Glu96Gly, Glu170Arg, Glu170Lys and/or Lys252Leu.

In addition to the at least one dehydrogenase, the reagent compositions can include at least one redox cofactor. As used herein, “redox cofactor” means a molecule that can serve as an acceptor for enzymatically transferred redox equivalents such as H⁻. As used herein, “redox equivalents” relates to a concept commonly used in redox chemistry that is well known to one of skill in the art. In particular, it relates to electrons that are transferred from a substrate of the dehydrogenase to the redox cofactor or electrons transferred to the indicator reagent from the redox cofactor.

In some instances, the redox cofactor can be PQQ, NAD, FAD or derivatives thereof. It will be understood that the redox cofactor included in the reagent compositions herein depends on the properties of the at least one dehydrogenase. For example, PQQ can be combined with a PQQ-dependent glucose dehydrogenase, NAD can be combined with a NAD-dependent glucose dehydrogenase, and FAD can be combined with a FAD-dependent glucose dehydrogenase. NAD derivatives (e.g., NAD/NADH and/or NADP/NADPH derivatives) include carbaNAD (cNAD). See, Intl Patent Application Publication No. WO 2007/012494.

Likewise, the reagent compositions can include at least one agent capable of eliciting a change in at last one measureable property of an indicator reagent in the presence of redox equivalents. As used herein, “agent capable of eliciting a change in at least one measurable property of an indicator reagent in the presence of redox equivalents” or “agent” means a molecule that, in the presence of the redox equivalents, can induce a change in at least one measurable property in an indicator reagent. It is to be understood that the at least one agent also may elicit a change in more than one measurable property of the indicator reagent, which may then subsequently be detected. Furthermore, the at least one agent may elicit a change in measurable properties of more than one indicator reagent, which may then subsequently be detected.

In this manner, the at least one agent is capable of transferring directly or indirectly via a further mediator, redox equivalents from the redox cofactor to the indicator reagent. As a consequence of the transfer of the redox equivalents, the indicator reagent will be modified such that a change in at least one measurable property occurs. In case the measurable property is an optical property such as, for example, a color-less or non-fluorescing indicator reagent in an oxidized state may be converted into a colored or fluorescent indicator reagent by the transfer of redox equivalents mediated by the agent in a reduced state. The transfer of the redox equivalents may be direct in that the redox equivalents are transferred by the at least one agent to the indicator reagent or may be indirect. In the latter case, the redox equivalents are transferred from the at least one agent to an intermediate mediator that subsequently transfers the redox equivalents to the indicator reagent.

It is understood that more than one mediator can be used. For example, the at least one agent may transfer the redox equivalents to a first mediator that subsequently transfers the redox equivalents to a second mediator, and the second mediator then transfers the redox equivalents to the indicator reagent. It further is understood that in such a mediator cascade more than two mediators could be used. An advantage of using one or more mediators for the transfer of the redox equivalents to the indicator reagent is that the timing of the detection of the measurable property can be improved.

In some instances, the at least one agent can be potassium ferricyanide, quinone derivatives, Nile blue (CAS No. 3625-57-8), Meldola's blue (CAS No. 7057-57-0), osmium complexes as disclosed in EP Patent No. 1 457 572, or transition metal complexes such as ruthenium hexamine chloride.

Another agent that can be used in the reagent composition is a phenazine such as, for example, phenazinethosulfate, phenazinmethosulfate, 1-(3-carboxypropoxy)-5-ethylphenaziniumtrifluoromethansulfonate, or 1-methoxyphenazine-methosulfate. These phenazines can be applied for eliciting a change in at least one optical property of an indicator reagent when optical detection methods are used. Details for the detection and on how phenazines can be applied can be found in EP Patent Application Publication No. 0 654 079.

Another agent that can be used in the reagent composition is a chinone such as, for example, phenanthrenchinone, phenanthrolinchinone, or benzo[h]-chinolinchinone.

Yet another agent that can be used in the reagent composition is a nitrosoaniline such as, for example, [(4-nitrosophenyl)imino]dimethanol-hydrochloride.

Moreover, the at least one agent can be an enzyme capable of eliciting a change in at least one measurable property of an indicator reagent in the presence of redox equivalents, where the enzyme catalyzes the transfer of redox equivalents from the redox cofactor to the indicator reagent.

As used herein, “measurable property” means any property of the reagent composition that changes in the presence of the analyte and that can be transferred into a physical signal of any kind. Generally, the change of the measurable property and/or the signal that can be generated therefrom are proportional to the analyte concentration in the sample.

In some instances, the measurable property is an electrochemical property. Thus, it is envisaged that the reagent composition includes one or more chemical reagents for reacting with the analyte to produce an electrochemical signal that represents the presence of the analyte in the sample fluid. Generally, electrochemical properties include amperometric or coulometric responses indicative of the analyte concentration. See, e.g., U.S. Pat. Nos. 5,108,564; 4,919,770; and 6,054,039.

In other instances, the measurable property is an optical property (i.e., a property which can be detected by an optical instrument). As used herein, “optical property” means a property of the indicator reagent that can be optically detected such as light absorption or emission, remission, refraction or polarization and properties associated therewith. It is understood that such a change of at least one optical property as used herein encompasses detecting presence of a property that was not detectable before, detecting absence of a property that has been detected before, and detecting quantitative changes of a property (i.e., detecting the change of the signal strength that correlates to the extent of the change of the at least one optical property). Examples of optical properties include, but are not limited to, color, fluorescence, luminescence, and refractometry.

The optical properties that are changed by the agent depend on the type of indicator reagent. Depending on the desired optical property to be detected and the agent to be used in the reagent composition, one of skill in the art is capable of selecting, without further ado, a suitable indicator reagent. Moreover, methods of converting the optical property as defined above into a physical signal that can be read as a measurement value are well known in the art and are described in, for example, EP Patent Nos. 0 821 234 and 0 974 303, as well as US Patent Application Publication No. 2005/0023152.

In view of the at least one agent, the reagent compositions can include at least one indicator reagent. As used herein, “indicator reagent” means a molecule or molecular entity that as a consequence of the transfer of redox equivalents will be modified such that a change in at least one measurable property occurs. Generally, indicator reagents are known in the art which, as a consequence of the transfer of redox equivalents, will be modified such that a change in at least one electrochemical property occurs. See, e.g., “Methods of Enzymatic Analysis” 2.6.(I), p.197ff (H. G Bergmeyer ed., Weinheim 1983).

In some instances, the at least one indicator reagent can be a heteropoly acid such as 2,18-phosphoromolybdenic acid; and chinones such as resazurine, dichlorophenolindophenole and/or tetrazolinum salts (e.g., commercially available WST-3, WST-4 and WST-5 salts; Dojindo, Inc. US). These indicator reagents are reduced upon transfer of redox equivalents, which is accompanied by a change in at least one optical property such as color.

In other instances, the at least one indicator reagent can be a fluorophore, the fluorescence of which is changed upon transfer of redox equivalents. Examples of fluorophores include, but are not limited to, flavin nucleotides and nicotinamide-adenine-dinucleotides referred to herein also in the context of the redox cofactors. If a redox cofactor such as carbaNAD or NAD is applied in the reagent compositions as an indicator reagent, the redox cofactor, agent and indicator reagent all may be represented by the same molecule (i.e., cNAD or NAD). Accordingly, these components may be represented in the reagent compositions by the same chemical entity. Moreover, a modified nitrosoaniline as disclosed in EP Patent Nos. 0 620 283 or 0 831 327, can be used as the agent and indicator reagent. Thus, the agent and indicator reagent may be represented in the reagent composition by the same chemical entity.

Moreover, the reagent compositions include at least one pyridine-carboxylic acid, derivative or salt thereof. As used herein, “pyridine-carboxylic acid” means those pyridine-mono- and dicarboxylic acids and derivatives and salts thereof known in the art. Regardless, the pyridine-carboxylic acid or derivative thereof has an activity of attenuating or preventing a reduction of enzymatic activity of at least one enzyme as detailed herein below.

Examples of pyridine-carboxylic acid include, but are not limited to, a pyridine-dicarboxylic acid such as 2,4-pyridinedicarboxylic acid (lutidinic acid), 2,5-pyridinedicarboxylic acid (isocinchomeronic acid), 2,6-pyridinedicarboxylic acid (dipicolinic acid) 2,3-pyridinedicarboxylic acid (quinolinic acid), 3,4-pyridinedicarboxylic acid, 3,5-pyridinedicarboxylic acid, or derivatives thereof. In some instances, the pyridine-carboxylic acid can be a pyridine-monocarboxylic acid or a derivative thereof. In other instances, the pyridine-carboxylic acid can be a pyridine-3-carboxylic acid (e.g., nicotinic acid, niacin, vitamin B3, CAS No. 59-67-6), pyridine-4-carboxylic acid (isonicotinic acid, CAS No. 55-22-1), pyridine-2-carboxylic acid (picolinic acid, CAS No. 98-98-6), or a sodium salt or a potassium salt thereof.

As used herein “derivative” of a pyridine-carboxylic acid means a structurally related organic molecule having the activity of attenuating or preventing a reduction of enzymatic activity of at least one enzyme as detailed herein below. The attenuation or reduction of the enzymatic activity is prevented in a similar or the same manner and/or to a similar or the same extent as found for the pyridine-carboxylic acid. The aforementioned derivatives can be obtained by chemically derivatizing one of the aforementioned pyridine-carboxylic acids in vitro by standard protocols.

In the reagent compositions, the pyridine-carboxylic acid, derivative or salt thereof attenuates or otherwise reduces a decrease of the enzymatic activity of the at least one enzyme under dry or humid conditions. In some instances, and as noted above, the at least one enzyme is the dehydrogenase. The at least one enzyme, however, also can be both the dehydrogenase and the agent. Moreover, the decrease of the enzymatic activity is prevented or at least significantly attenuated in the reagent compositions during manufacture and/or storage at room temperature or even higher temperatures as specified below by the at least one pyridine-carboxylic acid, derivative or salt thereof when compared to a control composition without the at least one pyridine-carboxylic acid, derivative or salt thereof, such that at least about 40%, at least about 50%, at least about 60%, at least about 70%, at least about 80%, or at least about 90% of the enzymatic activity of one or both enzymes is maintained. As used herein, “about” means within a statistically meaningful range of a value or values such as, for example, a stated concentration, length, width, height, angle, weight, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

In some instances, the residual activity is still present after about 2, 3, 4, 5, 6, 7, 8, 9, 10, 11, 12, 13, 14, 15, 16, 17, 18, 19, 20, or 25 weeks. Thus, for example, at least about 60% of the enzymatic activity of one or both enzymes is maintained after about 2, 3, 4, 5, 6, 7, 8, 9, 10, 11, 12, 13, 14, 15, 16, 17, 18, 19, 20, or 25 weeks; or at least about 70% of the enzymatic activity of one or both enzymes is maintained after about 2, 3, 4, 5, 6, 7, 8, 9, 10, 11, 12, 13, 14, 15, 16, 17, 18, 19, 20, or 25 weeks; or at least about 80% of the enzymatic activity of one or both enzymes is maintained after about 2, 3, 4, 5, 6, 7, 8, 9, 10, 11, 12, 13, 14, 15, 16, 17, 18, 19, 20, or 25 weeks; or at least about 90% of the enzymatic activity of one or both enzymes is maintained after about 2, 3, 4, 5, 6, 7, 8, 9, 10, 11, 12, 13, 14, 15, 16, 17, 18, 19, 20, or 25 weeks.

One of skill in the art understands that the residual activity of an enzyme stored for an extended time period depends on several factors, some of which are described herein. For example, the residual activity may depend on duration of storage, temperature, humidity, oxidative stress, irradiation, and the like. Here, it is shown that the pyridine-carboxylic acid or derivative thereof attenuates or reduces a decrease of the enzymatic activity of at least one enzyme in the reagent compositions under dry conditions.

As used herein, “dry conditions” means conditions where the reagent compositions are stored under and typically equilibrated with an atmosphere having a relative humidity of about 0% to about 50%, about 10% to about 50%, about 20% to about 50%, about 30% to about 50%, about 0% to about 40%, about 0% to about 30%, or about 0% to about 20%.

Likewise, and as used herein, “humid conditions” means conditions where the reagent compositions are stored under and typically equilibrated with, an atmosphere having a relative humidity of about 50% to about 100%, about 50% to about 90%, about 50% to about 85%, about 60% to about 100%, about 75% to about 100%, about 85% to about 100%, about 95% to about 100%, or about 85% to about 90%.

Moreover, the at least one enzyme in the reagent compositions maintains its aforesaid residual activity upon storage at more than about 0° C., more than about 5° C., more than about 10° C., more than about 20° C., more than about 30° C., or more than about 40° C. for at least one of the time frames and at least one of the humidity ranges as referenced above. Furthermore, the at least one enzyme in the reagent compositions maintains its aforesaid residual activity upon storage at most about 30° C., at most about 35° C., at most about 40° C., at most about 45° C., or at most about 50° C. for at least one of the time frames and at least one of the humidity ranges as referenced above. One of skill in the art understands that the temperature ranges recited above can be combined to closed value ranges (e.g., the at least one enzyme in the reagent compositions maintains its aforesaid residual activity upon storage at about 0° C. to about 30° C., at about 0° C. to about 35° C., at about 0° C. to about 40° C., at about 0° C. to about 45° C., at about 0° C. to about 50° C., at about 5° C. to about 30° C., at about 5° C. to about 35° C., at about 5° C. to about 40° C., at about 5° C. to about 45° C., at about 5° C. to about 50° C., at about 10° C. to about 30° C., at about 10° C. to about 35° C., at about 10° C. to about 40° C., at about 10° C. to about 45° C., at about 10° C. to about 50° C., at about 20° C. to about 30° C., at about 20° C. to about 35° C., at about 20° C. to about 40° C., at about 20° C. to about 45° C., at about 20° C. to about 50° C., at about 30° C. to about 35° C., at about 30° C. to about 40° C., at about 30° C. to about 45° C., at about 30° C. to about 50° C., at about 40° C. to about 45° C., or at about 40° C. to about 50° C. for at least one of the time frames and at least one of the humidity ranges as referenced above).

In particular, the pyridine-carboxylic acid, derivative or salt thereof attenuates or reduces a decrease of the enzymatic activity of the at least one dehydrogenase in the reagent compositions under humid conditions.

One of skill in the art understands that in certain instances a change in one of the above referenced conditions may have an impact on the effect of a second condition (e.g., an increase in temperature may aggravate the effect of increasing humidity on the stability of the at least one dehydrogenase). In some instances, the pyridine-carboxylic acid, derivative or salt thereof significantly attenuates or reduces a decrease of the enzymatic activity of at least one dehydrogenase in the reagent compositions stored under and equilibrated with, for example, a temperature of about 20° C. to about 70° C. and a relative humidity of about 60% to about 100%. In other instances, the pyridine-carboxylic acid, derivative or salt thereof significantly attenuates or reduces a decrease of the enzymatic activity of the at least one dehydrogenase in the reagent compositions stored under and equilibrated with a temperature of about 25° C. to about 60° C. and a relative humidity of about 70% to about 100%. In particular instances, the pyridine-carboxylic acid, derivative or salt thereof significantly attenuates or reduces a decrease of the enzymatic activity of the at least one dehydrogenase in the reagent compositions stored under and equilibrated with a temperature of about 30° C. to about 55° C. and a relative humidity of about 80% to about 95% or with a temperature of about 35° C. to about 45° C. and a relative humidity of about 85% to about 90%.

To provide the effect on the at least one dehydrogenase, the pyridine-carboxylic acid, derivative or salt thereof can be present in amounts of at least about 0.4 (w/w) %, at least about 0.8 (w/w) %, at least about 1.7 (w/w) %, at least about 3 (w/w) %, at least about 4 (w/w) %, at least about 5 (w/w) %, at least about 6 (w/w) %, at least about 7 (w/w) %, or at least about 8 (w/w) %.

In addition to the above, the reagent compositions can include other components. For example, the reagent compositions also can include at least one detergent, swelling agent, film-forming agent, oxidizing agent and/or solid particle. Suitable stabilizers, detergents, swelling agents, film forming agents, oxidizing agents, and/or solid particles for use in the reagent compositions are well known to one of skill in the art and need not be described in detail herein.

When included, the at least one detergent can be sodium-N-methyl-N-oleoyltaurat, N-octanoyl-N-methyl-glucamid, Mega 8 (N-methyl-N-octanoylglucamide), dioctylsodium sulfosuccinate (DONS), or Rhodapex® (e.g., CO-433 or CO-436).

When included, the at least one swelling agent can be methyl vinyl ether maleic acid anhydride copolymer, xanthan gum, or methyl vinyl ether maleic acid copolymer.

When included, the at least one film-forming agent can be a polyvinylpropionate dispersions, polyvinyl esters, polyvinyl acetates, polyacrylic esters, polymethacrylic acid, polyvinyl amides, polyamides, polystyrene, or mixed polymerizates such as those having butadiene, styrene or maleic acid ester.

When included, the at least one solid particle can be silica particles such as silicon dioxide, sodium silicates or aluminum silicates, kieselguhr, metal oxides such as titanium oxide and/or aluminum oxide, synthetic oxide materials, especially nanoparticles of oxide materials such as nanoparticles of silicon dioxide, aluminum oxide or titanium oxide, kaolin, powder glass, amorphous silica, calcium sulfate, and barium sulfate.

In particular instances, the reagent compositions include the components listed in Table 1 in the accompanying Examples.

As the reagent compositions generally are used in a dry state, procedures for removing solvent therefrom and, in particular, heat treatment, can affect the enzymatic activity of sensitive enzymes such as dehydrogenases. Moreover, under dry conditions, enzymes such as dehydrogenases tend to become more sensitive to oxidation processes and accompanying enzyme denaturation. See, Andersson (2000) Biotechnol. Appl. Biochem. 32:145-153. Accordingly, the manufacture of complex, dry reagent compositions for enzymatic detection assays, as well as the storage thereof, is often accompanied by increasing inactivation of the enzymes by denaturation, aggregation or other processes. Upon reconstitution of the enzymes in a solvent-containing surrounding, a reduced enzymatic activity is often observed.

Diagnostic Test Elements and Methods of Making the Same

Diagnostic test elements incorporating the inventive concept can include the reagent compositions described herein, where such test elements can be used for determining an analyte concentration or presence in a body fluid sample. The test elements also can include a carrier to which the reagent compositions can be applied or incorporated.

As used herein, “carrier” means a solid support onto which the reagent compositions can be applied. In some instances, the reagent compositions can be immobilized on the carrier; however, it is contemplated that the reagent compositions also can be spatially arranged on the carrier. Regardless, the carrier must be arranged in a manner as to allow for detecting a change of the at least one measurable property of the indicator reagent. That is, the carrier should not include components or a spatial arrangement that would interfere with detecting the at least one measurable property. Suitable carriers may include vials containing the reagent composition (e.g., vials arranged in a well-plate format). Other assays may apply optical waveguides or semiconductor plates. Other carriers, however, include solid supports used for test elements, such as test strips, where such test strips typically have one or more layers forming the solid carrier.

In some instances, the test elements include at least one test field containing the reagent compositions, where the at least one test field has a sample application side onto which the body fluid sample can be applied and a detection side that allows for detecting a change in a measurable property when an analyte of interest reacts with the reagent compositions. It is contemplated that cells, such as erythrocytes, that may be present in body fluid sample such as blood, do not reach the detection side.

Details on test element designs for use herein and the manufacture thereof can be found in, for example, EP Patent No. 0 821 234. Further test element designs for use herein can be found in EP Patent Nos. 1 035 919 and 1 035 920.

The at least one test field can include a transparent foil onto which one or more film layers are applied. The film layers can be produced from dispersions or emulsions of polymeric film formers. Dispersion film formers contain microscopic polymer particles that are insoluble in the carrier liquid (usually water) and are finely dispersed in the carrier liquid. If the liquid is removed by evaporation during film formation then the particles come closer and finely touch one another. The large forces that occur in this process and the gain in surface energy that accompanies film formation result in the particles growing into a substantially closed film layer. Alternatively, it is possible to use an emulsion of the film former that is dissolved in a solvent. The dissolved polymer is emulsified in a carrier liquid that is immiscible with the solvent. Examples of suitable film formers include, but are not limited to, polyvinyl esters, polyvinyl acetates, polyacrylic esters, polymethacrylic acid, polyacrylamides, polyamides and polystyrene. In addition to homopolymers, mixed polymerizates also can be used such as butadiene, styrene or maleic acid ester.

By adding a swelling agent that swells well (i.e., a substance that increases its volume when it takes up water), one not only obtains layers that can be penetrated relatively rapidly by the fluid sample but also obtains good cell and pigment separation properties despite this opening effect of the swelling agent. The swelling properties should be so good that for a test in which the change of the at least one measurable property is mainly dependent on the penetration of the sample fluid through the layer, the change of the measurable property is measurable after a maximum of one minute. Examples of suitable swelling agents include, but are not limited to, methyl vinyl ether maleic acid anhydride copolymer, xanthan gum and methyl vinyl ether maleic acid copolymer.

Single layer layouts of test elements are disclosed in EP Patent Nos. 1 566 637 and 1 780 288; however, two-layer layouts are contemplated. Two-layer layouts typically include a first and a second film layer resting on top of one another in this order. In this manner, it is important that the first layer located on the transparent foil scatters light considerably less than the overlying second layer. The non-coated side of the transparent foil is referred to as the detection side, and the side of the second layer that is opposite to the side with which the second layer rests on the first layer is referred to as the sample application side. The two so-called film layers are located on a transparent foil in the test field of the carrier, especially plastic foils that are impermeable to liquid. For example, polycarbonate foil has proven to be particularly suitable.

The two film layers can be produced from coating compounds that contain the same polymeric film formers, or they can be produced from coating compounds that in different polymeric film formers. Whereas the first layer includes a swelling agent and optionally a weakly light scattering filler, the second layer requires a swelling agent and in any case at least one pigment that scatters light strongly. In addition the second layer also can include non-porous fillers as well as porous fillers.

In the case of a single-layer film, the reagent compositions are included or incorporated in the single layer. In the case of multi-layer films, it is possible that the reagent compositions are included or incorporated in one film layer, such as the first film layer. However, it is also possible that the reagent compositions are included or incorporated in two or more film layers.

To optimize the test field in the carrier, it has proven to be particularly advantageous when all film layers, especially both layers in the case of two-layer layouts, contain a non-hemolyzing wetting agent. Neutral (i.e., non-charged wetting) agents should be used such as N-octanoyl-N-methyl glucamide.

To produce a two-layer test field for test elements, the respective film layers are each produced successively from a homogeneous dispersion of the components. For this, the transparent foil is used as a base to form the coating compound for the first film layer. After the coating compound for the first film layer has been applied with a particular layer thickness, the layer is dried. Afterwards, the coating compound for the second layer is applied to this layer as a thin layer thickness and subsequently dried. The test field produced in this manner can be mounted on a supporting layer for better handling, those materials coming into consideration for such a layer that do not take up the liquid to be examined. These are so-called non-absorptive materials, plastic foils for example made of polystyrene, polyvinyl chloride, polyester, polycarbonate or polyamide. Metal foils or glass also can be used as supporting materials.

In some instances, the detection side of the test field that is to be observed and measured for a change in at least one optical property of the indicator reagent should be visible through the supporting layer to determine the analyte to be detected in the body sample. This can be achieved by a transparent supporting layer. However, it is also possible that the supporting layer has a perforation that is covered by the detection side of the test field. The detection side is then visible through the perforation. In particular, the test elements can have a hole in the supporting layer below the detection side of the test field through which the detection side of the test field can be observed. The hole has a somewhat smaller diameter than the smallest linear dimension of the test field so that the test field outside the hole rests on the supporting layer and can be attached there.

In some instances, the test elements include a capillary canal. See, e.g., EP Patent No. 1 035 921.

In some instances, the test elements are produced from a test element band. See, e.g., EP Patent No. 1 593 434.

In view of the above, methods incorporating the inventive concept can include methods making test elements that incorporate a reagent composition as described herein for use in diagnostics. The methods can include the steps described herein, and these steps may be, but not necessarily, carried out in the sequence as described. Other sequences, however, also are conceivable. Furthermore, individual or multiple steps may be carried out either in parallel and/or overlapping in time and/or individually or in multiply repeated steps. Moreover, the methods may include additional, unspecified steps.

Dry reagent compositions for use in the test fields of the test elements can be provided dissolving the components of the reagent composition first in a solvent or mixture of solvents and subsequently removing the solvent or mixture of solvents by a suitable treatment such that the remaining reagent composition is essentially free of the solvent or solvent mixture. Examples of suitable treatments for removing the solvent or mixture include, but are not limited to, heat treatment, evaporation techniques, freeze drying and the like. In some instances, the treatment is heat treatment and can be under the following conditions: heat treatment at about 60° C. or more for about 20 minutes to about 45 minutes or at about 95° C. for about 1 minute to about 2 minutes with heat circulation; thickness of the reagent composition of about 20 μm to about 200 μm or less; at a pressure of 1 bar or 0.1 bar. Moreover, it will be understood that to keep the reagent compositions under dry conditions, storage generally is carried out in the presence of a drying agent such as a desiccant. Examples of suitable drying agents include, but are not limited to, silica gel, zeolites, calcium carbonate or magnesium sulfate.

As used herein, “solvent” means a substance that dissolves components of the reagent compositions described herein under conditions that do not irreversibly impair the function of the components and, in particular, the enzymatic activity of the at least one dehydrogenase. Moreover, it is envisaged that the solvent dissolves the components under standard pressure such as, for example, 1 bar+/−10% within a temperature range of about 5° C. to about 50° C., room temperature, or even 20° C.+/−10° C. Examples of suitable solvents include, but are not limited to, water and alcohols, such as hexanol, 2-methoxy-propanol, 2-methyl-2-butanol. One of skill in the art understands that a solvent can be a mixture of two or more of the aforementioned solvents. Exemplary solvent mixtures include mixtures of water or water-based buffers with alcohols and in particular, the solvent mixtures referred to in the accompanying Examples below.

Methods of Analyte Detection

Methods incorporating the inventive concept can include methods of detecting an analyte concentration or presence in a fluid sample with test elements as described herein. The methods can include the steps described herein, and these steps may be, but not necessarily, carried out in the sequence as described. Other sequences, however, also are conceivable. Furthermore, individual or multiple steps may be carried out either in parallel and/or overlapping in time and/or individually or in multiply repeated steps. Moreover, the methods may include additional, unspecified steps.

The methods can begin by contacting a diagnostic test element as described herein with a body fluid sample having or suspected of having the analyte of interest under conditions suitable for transforming the at least one dehydrogenase of the reagent composition to a reconstituted state.

As noted elsewhere, depending on the substrate specificity of the dehydrogenase used in the dry reagent composition of the test field on the diagnostic test element, different analytes can be determined.

As used herein, “contacting” means that a body fluid sample is applied to the carrier or test element in a manner so as to allow for physical contact of the reagent composition and the body fluid sample. In particular, contacting can be carried out for a time and under conditions being sufficient for allowing the dehydrogenase to be reconstituted (i.e., wetted and dissolved), and thus, to become biologically active. Suitable conditions depend on the carrier and are known in the art. The body fluid sample applied to the test element can have a volume of less than about 2 μl or even less than 1 μl. Alternatively, the body fluid sample applied to the test element can have a volume of less than about 20 μl or even less than about 3 μl.

Upon reconstitution of the dehydrogenase to a biologically active state, the enzyme then binds to the analyte of interest in the body fluid sample and converts it into the respective product and redox equivalents. The redox equivalents generated by the dehydrogenase allow for determining the dehydrogenase activity since the redox equivalents generated by the enzymatic conversion catalyzed by the dehydrogenase are transferred by the agent capable of eliciting a change in at least one measurable property of the indicator reagent in the presence of redox equivalents in the composition comprised to the indicator reagent. The change in the at least one measurable property of the indicator reagent can then be measured. Depending on the diagnostic test element, the measurement of the change of the measurable property can be achieved by different techniques known in the art. See, e.g., Int'l Patent Application Publication Nos. WO 2012/010454A1 and WO 2007/115732A1, as well as U.S. Pat. No. 6,055,060.

In some instances, the measurable property is an optical property, and the detector includes at least one light source for illuminating test element and at least one optically sensitive element adapted to determine detection light from the test element. For detecting the change of an optical property, such as color, a spatially resolving optical detector may be used. As used herein, “spatially resolving optical detector” means an optical detector having a multiplicity of optical sensors that are able to record regions of the detection side of the detection layer (e.g., a CCD chip and/or CMOS chip). In addition, the spatially resolving optical detector can include at least one optical element for imaging the detection side and/or the detection layer onto an image-sensitive surface of the spatially resolving optical detector.

The methods also include measuring a change in at least one measurable property of the indicator reagent in the wetted reagent composition, whereby a presence or an amount of the analyte in the body fluid sample is determined. A change in at least one measurable property measured generally is indicative for the presence of the analyte. One of skill in the art understands that to determine the analyte concentration, it may be necessary to compare the extent of the change of the measurable property. To this end, it may be necessary to compare a detected signal accompanying the measurable change to signals accompanying measurable changes elicited by known amounts of analytes (i.e., calibration signals and/or reference values). How such a calibration can be established is well known to one of skill in the art.

As used herein, “body fluid sample” means all body fluids known or suspected to comprise the analyte to be determined. Examples of suitable body fluid samples include, but are not limited to blood including whole blood, plasma and serum, urine, saliva, liquor, synovial liquid, and sudor. Typically, the body fluid sample is a whole blood sample.

As used herein, “analyte” means a biological molecule present in the body fluid sample, the presence, absence or amount of which shall be determined in accordance with a known detection method. Since the determining described herein is based on the enzymatic activity of a dehydrogenase, it will be understood that the analyte of interest is a substrate of the dehydrogenase comprised by the composition. Examples of suitable analytes include, but are not limited to glucose, maltose, mannose, galactose, glutamate, glucose-6-phosphate, ethanol or lactose.

As used herein “amount” means an absolute or relative amount of an analyte present in a body fluid sample applied to a test element. A relative amount is the concentration (i.e., the amount in relation to the volume).

Methods of Attenuating, Reducing or Preventing a Decrease of Enzymatic Activity of an Enzyme Such as a Dehydrogenase Under Dry and Humid Conditions

Methods incorporating the inventive concept can include methods of attenuating, reducing or preventing a decrease of enzymatic activity of a dehydrogenase in a reagent composition as described herein under dry or humid conditions. The methods can include the steps described herein, and these steps may be, but not necessarily, carried out in the sequence as described. Other sequences, however, also are conceivable. Furthermore, individual or multiple steps may be carried out either in parallel and/or overlapping in time and/or individually or in multiply repeated steps. Moreover, the methods may include additional, unspecified steps.

The methods can begin by contacting at least one pyridine-carboxylic acid, derivative or salt thereof with a dehydrogenase under dry or humid conditions. In some instances, at least one pyridine-carboxylic acid, derivative or salt thereof and the dehydrogenase are included as components of a reagent composition, especially a reagent composition on a test element. The at least one pyridine-carboxylic acid, derivative or salt thereof is provided in an amount sufficient to attenuate, reduce or prevent a decrease of the enzymatic activity of the at least one dehydrogenase. In some instances, it is advantageous to contact the at least one pyridine-carboxylic acid, derivative or salt thereof with the dehydrogenase when removing a solvent from a reagent composition by heat treatment and/or while maintaining the reagent composition under dry conditions as part of the test element.

### EXAMPLES

The inventive concept will be more fully understood upon consideration of the following non-limiting examples, which are offered for purposes of illustration, not limitation.

### Example 1

### Preparing Test Elements Having a Dry Reagent Composition

Methods: different reaction films for determining glucose levels were generated and coated on a foil essentially as described in EP Patent Application Publication No. 0 821 234, Example 1. The formulation of the composition is shown below in Table 1.

TABLE 1
Components per 100 g of the first coating film prior to drying.

| Component | Amount | | | | |
|---|---|---|---|---|---|
| GDH from Bacillus | 1.09 g | 1.09 g | 1.09 g | 1.09 g | 1.09 g |
| subtilis | | | | | |
| diaphorase from | 0.77 g | 0.77 g | 0.77 g | 0.77 g | 0.77 g |
| B. subtilis | | | | | |
| NAD | 0.58 g | 0.58 g | 0.58 g | 0.58 g | 0.58 g |
| Na/K phosphate buffer or | 0.35 g | 0.35 g | 0.35 g | 0.35 g | 0.35 g |
| HEPES | | | | | |
| nicotinic acid (resp. | 0.00 g | 1.0 g | 1.5 g | 2.5 g | 3.5 g |
| picolinic acid or | | | | | |
| isonicotinic acid) | | | | | |
| xanthan gum | 0.29 g | 0.29 g | 0.29 g | 0.29 g | 0.29 g |
| silica FK 320DS | 5.80 g | 5.80 g | 5.80 g | 5.80 g | 5.80 g |
| sodium-N-methyl-N- | 0.03 g | 0.03 g | 0.03 g | 0.03 g | 0.03 g |
| oleoyl-taurate | | | | | |
| N-octanoyl-N-methyl- | 0.17 g | 0.17 g | 0.17 g | 0.17 g | 0.17 g |
| glucamide | | | | | |
| polyvinylpyrrolidone | 0.86 g | 0.86 g | 0.86 g | 0.86 g | 0.86 g |
| tetraethylammonium- | 0.07 g | 0.07 g | 0.07 g | 0.07 g | 0.07 g |
| chloride | | | | | |
| 2,18- | 0.33 g | 0.33 g | 0.33 g | 0.33 g | 0.33 g |
| phosphormolybdenic | | | | | |
| acid hexasodium salt | | | | | |
| polyvinylpropionate- | 5.00 g | 5.00 g | 5.00 g | 5.00 g | 5.00 g |
| dispersion (50 Gew.- | | | | | |
| % in water) | | | | | |
| K3[Fe(CN)6] | 0.01 g | 0.01 g | 0.01 g | 0.01 g | 0.01 g |
| 2-methyl-2 butanol | 1.00 g | 1.00 g | 1.00 g | 1.00 g | 1.00 g |
| Add water up to 100 g | | | | | |

A pH of 6.8 was adjusted and the composition was coated as a film (about 120 μm) on a polycarbonate foil (125 μm). The coated composition was subsequently dried at 50° C.

TABLE 2
Components of the second coating film.

| Component | Amount |
|---|---|
| Gantrez | 1.47 g |
| sodium-N-methyl-N-oleoyl-taurate | 0.03 g |
| PVP K25 | 2.01 g |
| Mega 8 | 0.37 g |
| tetraethylammoniumchloride | 0.45 g |
| silica FK 320DS | 2.00 g |
| titanium dioxide E171 | 22.00 g |
| polyvinylpropionate-dispersion | 6.25 g |
| (50 w % in water) | |
| bis-(2-hydroxyethyl)-(4- | 0.48 g |
| hydroximinocyclohexa-2,5-dienylidin)- | |
| ammoniumchloride | |
| 2,18-Phosphormolybdenic acid | 1.41 g |
| hexasodium salt | |
| K3[Fe(CN)6] | 0.01 g |
| 2-Methyl-2 butanol | 1.00 g |
| add water up to 100 g | |

The pH was adjusted to 6.8, and the composition was coated as a second film (about 25 μm) onto the first film coated on the foil. The coated composition was subsequently dried at 50° C. Test elements for glucose determination were generated as described in paragraphs [0063]-[0067] of EP Patent Application Publication No. 0 821 234.

### Example 2

### Determining Enzymatic Activity in Test Elements After Storage

Methods: test elements were stored in plastic vials in the presence of a desiccant at 4° C. and 45° C. for 3 and 9 weeks.

In a subsequent step, the reagent pads of five test elements were eluted by ultra-sonication using elution buffer. In the supernatant, enzymatic activity was determined, and mean value was calculated. The elution buffer was as follows: Tris/HCl, NaCl, NAD; pH 8.5; and the detection technique was as follows: UV-detection at 340 nm.

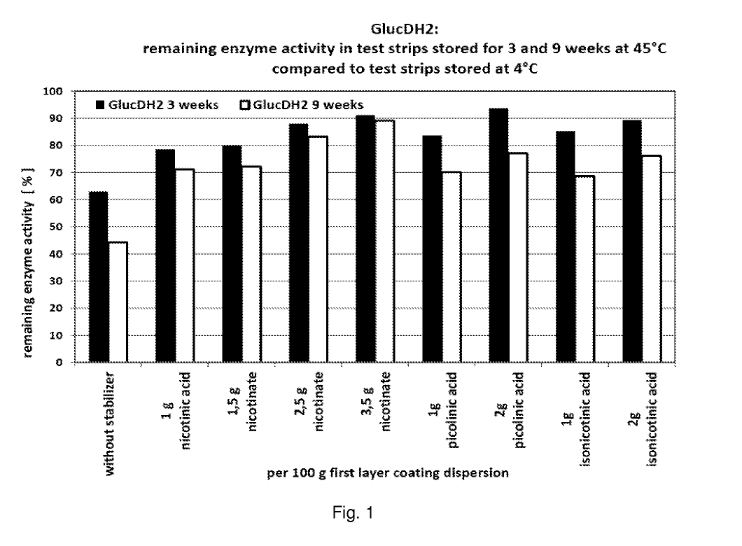

Results: FIG. 1 shows that there is a significant stabilizing effect (i.e., more than 10%) with nicotinic acid, picolinic acid and isonicotinic acid.

### Example 3

### Determining Test Element Function After Different Storage Conditions

Methods: test elements were stored in plastic vials in the presence of a drying agent for 9 weeks at 4° C., 24° C., 35° C. and 45° C. Test elements were used to determine blood glucose levels in a plurality of venous blood samples. The samples were measured with a reference method (Hitachi) in parallel. Results were normalized with respect to the reference samples.

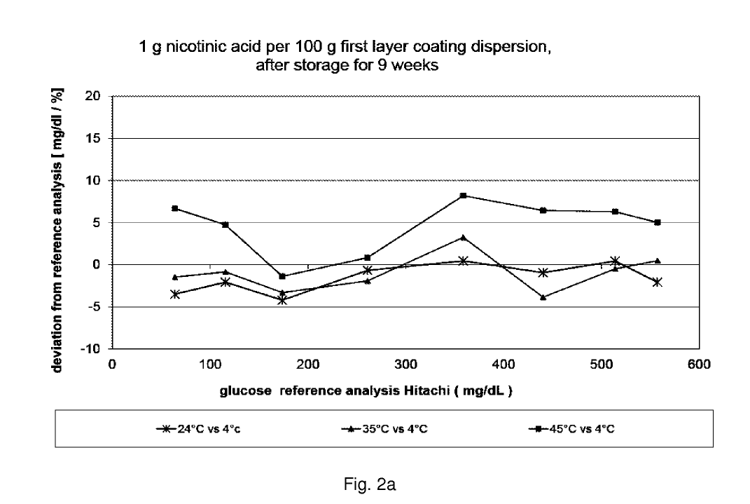

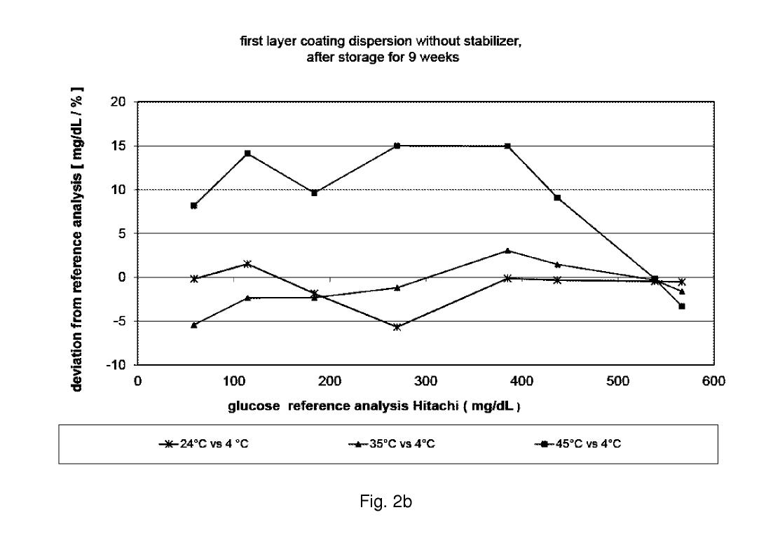

Results: deviation of the indicated glucose values from reference is shown in FIG. 2a (with nicotinic acid) and 2b (without added stabilizer). Each data point is a mean from 10 test elements. Test elements without added stabilizer stored at 45° C. show higher glucose values in the range of 100 mg/dL-400 mg/dL glucose (deviations >10%), whereas test elements with nicotinic acid show deviations within ±8%.

### Example 4

### Stabilization of Glucose Dehydrogenase Mutant 2 in the Presence of the Coenzyme, cNAD

Methods: test elements with added nicotinic acid were prepared and analog to Example 1, but with adding coenzyme cNAD (see, Int'l Patent Application Publication No. WO 2007/012494) instead of NAD (same amount on molar basis).

As cNAD itself is much more stable than NAD, more challenging conditions for the test elements were chosen. Consequently, test elements were stored: (a) in open vials at 85% r.h. and 35° C., or (b) in closed vials with desiccant at 35° C.

Analysis of enzymatic activity was the same as above in Example 2.

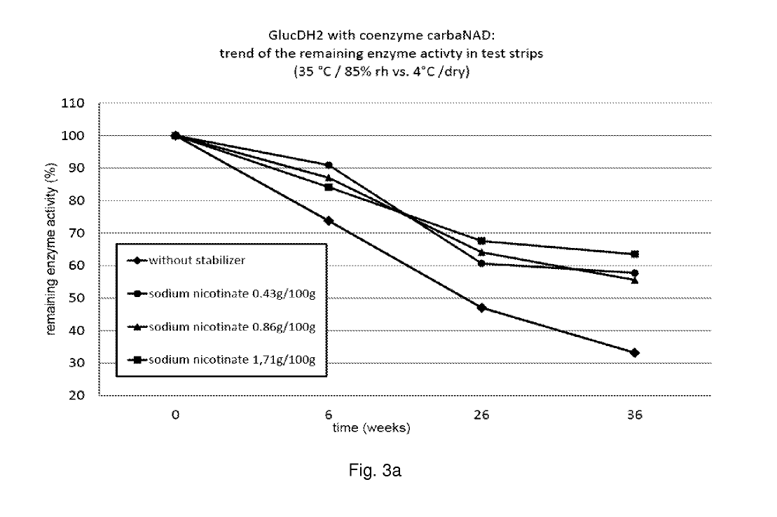

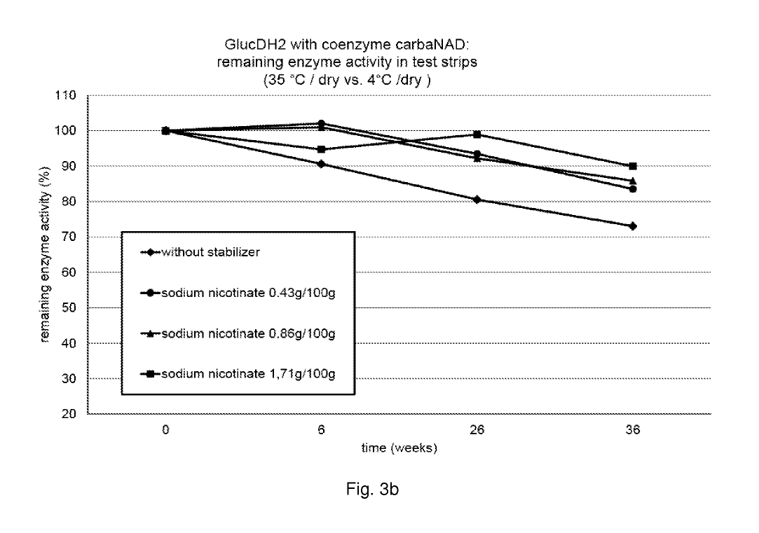

Results: FIG. 3a shows results from open vials at high humidity, and FIG. 3b shows results from closed vials. In the figures, there is a significant stabilizing effect with increasing amounts of nicotinic acid.

All of the patents, patent applications, patent application publications and other publications recited herein are hereby incorporated by reference as if set forth in their entirety.

The present inventive concept has been described in connection with what are presently considered to be the most practical and preferred embodiments. However, the inventive concept has been presented by way of illustration and is not intended to be limited to the disclosed embodiments. Accordingly, one of skill in the art will realize that the inventive concept is intended to encompass all modifications and alternative arrangements within the spirit and scope of the inventive concept as set forth in the appended claims.

The invention claimed is:

1. A dry composition comprising:
a dehydrogenase;
a redox cofactor;
an agent capable of eliciting a change in at least one measurable property of an indicator reagent in the presence of redox equivalents;
the indicator reagent; and
at least one pyridine-carboxylic acid, derivative or salt thereof, wherein the pyridine-carboxylic acid, derivative or salt thereof is a pyridine-monocarboxylic acid or a pyridine-dicarboxylic acid.

2. The composition of claim 1, wherein the dehydrogenase is a glucose dehydrogenase selected from the group consisting of glucose dehydrogenase, pyrrolo quinoline quinone (PQQ)-dependent glucose dehydrogenase, glucose-6-phosphate dehydrogenase, nicotinamide adenine dinucleotide (NAD)-dependent glucose dehydrogenase, flavin adenine dinucleotide (FAD)-dependent glucose dehydrogenase, and enzymatically active mutants thereof.

3. The composition of claim 1, wherein the pyridine-carboxylic acid is selected from the group consisting of pyridine-3-carboxylic acid, pyridine-4-carboxylic acid, pyridine-2-carboxylic acid, and sodium and potassium salts thereof.

4. The composition of claim 1, wherein the agent capable of eliciting a change in the at least one measurable property in the presence of redox equivalents can transfer redox equivalents from the redox cofactor to the indicator reagent.

5. The composition of claim 4, wherein the agent capable of eliciting a change in the at least one measurable property in the presence of redox equivalents is selected from the group consisting of a diaphorase, a phenazine, a nitrosoaniline; and a chinone.

6. The composition of claim 4, wherein the diaphorase is a lipoamide dehydrogenase or a NADH dehydrogenase, wherein the phenazine is phenazinethosulfate, phenazinmethosulfate, 1-(3-carboxypropoxy)-5-ethylphenaziniumtrifluoromethansulfonate or 1-methoxyphenazinmethosulfate, wherein the nitrosoaniline is [(4-nirosophenyl)imino]dimethanol-hydrochloride, and wherein the chinone is phenanthrenechinone, phenanthrolinchinone or benzo[h]-chinolinchinone.

7. The composition of claim 1, wherein the redox cofactor is selected from the group consisting of carbaNAD, NAD, FAD and PQQ.

8. The composition of claim 1, wherein the measurable property is an optical property.

9. The composition of claim 1, wherein the pyridine-carboxylic acid is in an amount sufficient to attenuate or prevent a decrease of enzymatic activity of the dehydrogenase under dry and/or humid conditions.

10. A diagnostic test element for determining an analyte concentration or presence in a body fluid sample, the diagnostic test element comprising:
gent composition of claim 1; and
a carrier, where the reagent composition is arranged on the carrier.

11. The diagnostic test element of claim 10, wherein the carrier comprises a test field comprising the reagent composition, and wherein the test field has a sample application side onto which the body fluid sample can be applied and a detection side that allows for detecting a change in at least one measurable property of the reagent composition when the analyte reacts with the reagent composition.

12. The diagnostic test element of claim 10, wherein the pyridine-carboxylic acid in in an amount sufficient to attenuate or prevent a decrease of enzymatic activity of the dehydrogenase in the reagent composition under dry and/or under humid conditions.

13. A method of manufacturing a diagnostic test element, the method comprising the step of:
generating a dry reagent composition of claim 1 on a carrier.

14. The method of claim 13, wherein the generating step comprises the steps of:
(i). applying a composition comprising all components of the reagent composition of claim 1 in a solvent to a test field on the carrier; and
(ii). removing the solvent from the applied composition to form the dry reagent composition; or
(i). applying a first composition comprising at least one dehydrogenase, at least one redox cofactor, at least one indicator reagent, and at least one pyridine-carboxylic acid, derivative or salt thereof in a solvent to a test field on the carrier;
(ii). removing the solvent from the first composition to form a first layer on the test field;
(iii.). applying a second composition comprising at least one agent capable of eliciting a change in at least one measurable property of an indicator reagent in the presence of redox equivalents and at least one pyridine-carboxylic acid, derivative or salt thereof in a solvent on the first layer; and
(iv). removing the solvent from the second composition to form a second layer; or
(i). applying a first composition comprising at least one dehydrogenase, at least one redox cofactor, at least one indicator reagent, and at least one pyridine-carboxylic acid, derivative or salt thereof in a solvent to a test field on a carrier;
(ii). removing the solvent from the first composition to form a first layer on the test field;
(iii). applying a second composition including at least one redox cofactor, at least one agent capable of eliciting a change in at least one measurable property of an indicator reagent in the presence of redox equivalents, and at least one indicator reagent in a solvent on the first layer; and
(iv). removing the solvent from the second composition to form a second layer; or
(i). applying a first composition comprising at least one dehydrogenase, at least one indicator reagent, and at least one pyridine-carboxylic acid, derivative or salt thereof in a solvent to a test field on a carrier;
(ii). removing the solvent from the first composition to form a first layer on the test field;
(iii). applying a second composition including at least one redox cofactor, at least one agent capable of eliciting a change in at least one measurable property of an indicator reagent in the presence of redox equivalents, at least one indicator reagent, and at least one pyridine-carboxylic acid, derivative or salt thereof in a solvent on the first layer; and
(iv). removing the solvent from the second composition to form a second layer; or
(i). applying a first composition including at least one agent capable of eliciting a change in at least one measurable property of an indicator reagent in the presence of redox equivalents, at least one indicator reagent, and at least one pyridine-carboxylic acid, derivative or salt thereof in a solvent to a test field on a carrier;
(ii.) removing the solvent from the first composition to form a first layer on the test field;
(iii). applying a second composition including at least one dehydrogenase, at least one redox cofactor, at least one indicator reagent, and at least one pyridine-carboxylic acid, derivative or salt thereof in a solvent on the first layer; and
(iv). removing the solvent from the second composition to form a second layer; or
(i). applying a first composition comprising at least one redox cofactor, at least one agent capable of eliciting a change in at least one measurable property of an indicator reagent in the presence of redox equivalents, at least one indicator reagent, and at least one pyridine-carboxylic, derivative or salt thereof in a solvent to a test field on a carrier;
(ii). removing the solvent from the first composition to form a first layer on the test field;
(iii). applying a second composition comprising at least one dehydrogenase, at least one redox cofactor, at least one indicator reagent, and at least one pyridine-carboxylic acid, derivative or salt thereof in a solvent the first layer; and
(iv). removing the solvent from the second composition to form a second layer; or
applying a first composition comprising at least one redox cofactor, at least one agent capable of eliciting a change in at least one measurable property of an indicator reagent in the presence of redox equivalents, at least one indicator reagent, and at least one pyridine-carboxylic acid, derivative or salt thereof in a solvent to a test field on a carrier;
(ii). removing the solvent from the first composition to form a first layer on the test element;
(iii). applying a second composition including at least one dehydrogenase, at least one indicator reagent, and at least one pyridine-carboxylic acid, derivative or salt thereof in a solvent on the first layer; and
(iv). removing the solvent from the second composition to form a second layer.

15. The method of claim 14, wherein the pyridine-carboxylic acid, derivative or salt thereof is in an amount to attenuate, reduce or prevent a decrease of enzymatic activity of the at least one dehydrogenase under dry and/or under humid conditions.

16. A method of determining a concentration or presence of an analyte in a body fluid sample, the method comprising the steps of:
contacting the diagnostic test element of claim 10 with the body fluid sample under conditions suitable to transform the dehydrogenase to its reconstituted state; and
measuring a change in at least one measurable property of the indicator reagent in the wetted reagent composition on the diagnostic test element to determine the concentration or presence of the analyte in the body fluid sample.
